(19) United States
(12) Patent Application Publication
(10) Pub. No.: US 2002/0144685 A1
(43) Pub. Date: Oct. 10, 2002

(54) Multipurpose device for preventing and treating snoring and sleep apnea and /or preventing gnashing of teeth

(76) Inventors: Bredov Vladimir Ivanovich, Moscow (RU); Ryazanov Evgeniy Alekseevich, Moscow (RU)

Correspondence Address: McDERMOTT, WILL & EMERY, 600 13th Street, N.W., Washington, DC, 20005-3096, US

(21) Appl. No.: 10/102,858

(22) Filed: Mar. 22, 2002

(30) Foreign Application Priority Data
Apr. 2, 2001 (RU) 2001108645

(51) Int. Cl. A61F 5/56

(52) U.S. Cl. 128/848; 128/861; 602/902

(57) ABSTRACT

The invention pertains to the medical devices and may be used as the method for prophylaxis and treating snoring and sleep apnea, and also for preventing gnashing of teeth during a sleep. The device contains joined through the connecting element the cup-shaped fixture and outer and inner restrictive petals with the holes. The petals are set up on the connecting element with an opportunity of moving along it and fixing on it.

The cup-shaped fixture has the section in the form of the arc in the surface of the longitudinal axis section of the connecting element in the area of its adjoining to the fixture, symmetrical to the planes of the petals of the fixture, and the section in the form of the parabola in the surface of the axis section, perpendicular to the identified.

The invention pertains to medical devices and is intended for treating snoring, sleep apnea syndrome (short pauses in breathing during a sleep), and also for preventing gnashing of teeth during a sleep.

During a sleep a human often can produce non-articulate sounds called snoring, caused by resonant vibrations during the oral cavity tissues and airflow interaction, when the airflow is going through between the palatine curtain and the tongue, and also between the route of the tongue and the posterior wall of the larynx. In the first case these sounds are caused most often by hypotonia of the muscles of the palatine curtain, in the second case, by tongue retraction.

Snoring sleep apnea is of great discomfort for the people around and for the snoring person it leads to appearing and developing of different diseases caused by breathing impairment. Sleep apnea can continue from several seconds to two minutes, the total time of such apneas may reach 4 hours during the night. The sequels of the multiple stops of breathing are blood and tissue gas disturbances (less saturation by oxygen) and of oxidation-reduction reactions in the tissues and organs of the organism. As a result those who snore do not have a good sleep and experience some sorts of malaise during the day.

Snoring and sleep apnea are especially dangerous for the elderly and those who is already over their middle age, because age-related organic changes in circulatory and nervous systems enhance the mentioned above phenomena. With the age increasing the snoring leads to the apnea intensification, heart disturbances, insult and infarction development and even to the sudden death caused by stop of breathing. It was established by the studies that the snoring is characteristic to a certain degree for the most people above 50 years old, and even people who never snored being young may begin snoring because of the age related changes in their organisms.

From the previous level of technique there is a well-known device for preventing snoring containing the wire frame with two springs on the opposite ends. SU No. 1697792, M., kl. A61 F 5/56, 1991.

This device is not comfortable in use and may lead to sudden swallowing of one of the springs.

There is also a well-known device for preventing snoring containing a biting-vestibular mouth-piece with the support for the tongue pressing. U.S. Pat. No. 39,711,370, kl. A61 F 5/56, 1976.

The device causes unpleasant feeling by using and, besides, an individual matching of the shape and size of the cap depending on the individual anatomic features is necessary.

There is also an anti-snoring device containing an internal working element, connected to external limiting element through the connecting element. U.S. Pat. No. 4,669,459, kl. A61 F 5/56, 1987.

The disadvantage of this device is the necessity of an individual matching of the elements for each patient, and it creates inconvenience by its mass production.

As the closest analogue to the proposed invention the authors have chosen the Russian utility model under certificate No. 10549 of Aug. 16, 1999. The multipurpose device for preventing and treating snoring and breathing stops and/or gnashing of teeth contains joined together by connecting strip a cup-shaped fixture of a patient's tongue tip and outer and inner concave restrictive petals set up with an opportunity of regulated moving along the ribbed surface of the connecting strip and fixing their position on it.

The disadvantage of the device is its insufficient comfort and efficiency of operation.

The technical result achieved in the proposed invention is in a higher level of comfort and efficiency of the device operation achieved by providing optimal static and dynamic load on the tongue.

The technical result is achieved by that in the device for preventing treating snoring and breathing stops and/or preventing gnashing of teeth, containing the joined together by a connecting element the cup-shaped fixture and inner restrictive petal with through holes, set up on the connecting element with an opportunity of moving along it and fixing on it, according to the invention, the curves made by the two mutually perpendicular section planes of the cup-shaped fixture, paralleled to the axis of the connecting element or passing through the axis and presented as the symmetry planes of the cup-shaped fixture, have different curvature, for instance, one of the curves may have a form of the parabola, the other—of the arc of the circle.

The longitudinal axis of the connecting element in the area of its adjoining to the fixture may coincide with the parabola axis or may be set off relatively to it.

In the first case the connecting element may have a step bend.

The angle between the tangents to the outer parabolic planes of the cup-shaped fixture, outgoing from the point situated on the axis of the parabola in the distance of 4-7 mm from the outer plane of the cup-shaped fixture, makes 30-90°.

The connecting element can be either shaped in the form of a strip of the rectangular cross section, or it can have an oval or circular cross section.

The device can be provided with the outer restrictive petal, set up on the connecting element with an opportunity of moving along it and fixing on it and having a through hole.

The dimensions and quantity of the holes in the identified petals are supposed to be chosen depending on the conditions of providing the laminarity of the airflow breathed in or out by a statistically average patient.

The square of the surface of the outer restrictive petal preferably exceeds the square of the surface of the inner one, and the square of each of the holes in the outer restrictive petal preferably exceeds the square of each of the holes in the inner restrictive petal.

By testing the proposed device it is established that, regardless of the properties of the material applied for the device manufacturing, there are certain proportions of the geometrical dimensions of the separate parts of the device, which correspond to the statistically average patient to the most extent, by those the device has the minimal weight and, consequently, the minimal specific consumption of materials, what leads to the patient's quick adaptation to the device.

The drawings of some specific, but not limiting the invention, type of the device are enclosed, where:

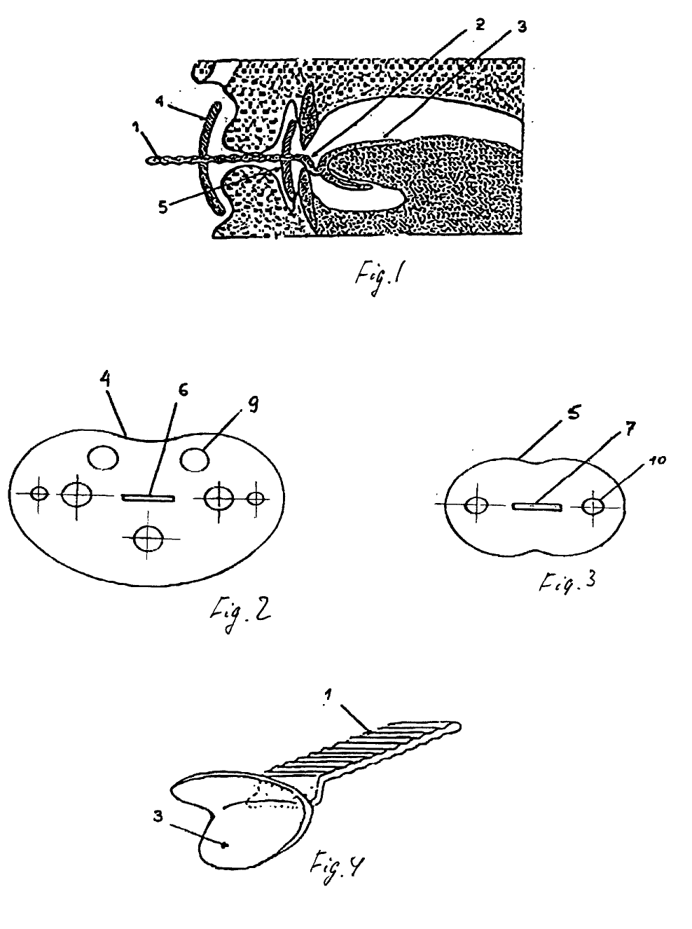

On the FIG. 1 there is the proposed device assembled and its installation scheme in the cavity of the mouth;

On the FIG. 2 the version of the outer restrictive petal design is shown;

On the FIG. 3 the version of the inner restrictive petal design is shown;

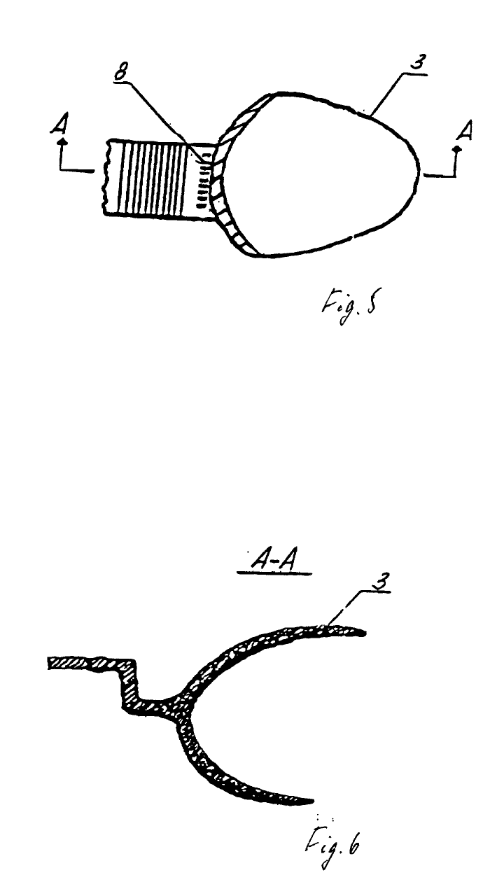

On the FIG. 4 the version of the design of the cup-shaped fixture with the connecting strip

On the FIGS. 5, 6—sections of the cup-shaped fixture.

The device for preventing and treating snoring and sleep apnea and/or preventing gnashing of teeth contains joined through the connecting element 1 with the springy step bend 2 the cup-shaped fixture 3 for the tongue tip and outer and inner restrictive petals 4 and 5.

The length of the connecting element 1 from the edge to the surface of the adjoining to the cup-shaped fixture 3 along the axis line makes 30 mm.

The distance from the surface of adjoining of the cup-shaped fixture 3 to the connecting element 1 to the outer surface of the bend 2 along the axis line makes not more than 0,3 of the width of the connecting element.

The height of the bend 2 in the single-planed measurement should be not less than 0,3 of the width of the connecting element 1.

The surface of the connecting element 1 is ribbed performed for providing the regulated moving and fixing on it of the outer and inner restrictive petals 4, 5, provided with the slots 6 and 7 corresponding to the form of the cross section of the connecting element 1. The bend 2 has longitudinal slots 8. The restrictive petals 4 and 5 are performed with the through holes 9 and 10. The square of each of the holes 9 in the outer restrictive petal 4 exceeds 2 times approximately the square of each of the holes 10 in the inner restrictive petal 5.

The cup-shaped fixture 3 has a form of the arc in the plane of the longitudinal axis section of the connecting element 1 (see FIG. 5) in the area of its adjoining to the fixture 3 symmetrical to the planes of the petals of the fixture. In the plane of the axis section, perpendicular to the identified, the cup-shaped fixture 3 has the form of the parabola (see. FIG. 6), and the angle between the tangents to the outer parabolic surfaces of the fixture, outgoing from the point, situated on the axis of the parabola at the distance of 5 mm from the outer surface of the fixture, makes 30-90°.

This type of the form of the cup-shaped fixture 3 provides the exclusion of overload on to the tongue, that enhances the comfort of the use of the device and also the efficiency of its operation.

The dimensions of the holes 9, 10 are chosen so that the airflow, breathed in and out by the patient, was laminar. This also adds to the comfort and efficiency of the device.

The cup-shaped fixture 3 and the restrictive petals 4, 5, and in the preferable variant also the connecting element 1 are made of elastic polymeric material. This increases convenience and comfort of its application.

The device works as follows:

The device is oriented in such a way that the cup-shaped fixture is directed towards the mouth of the patient, it is put in the mouth cavity until the cup-shaped fixture 3 captures the tip of the tongue (see FIG. 1). The outer and inner restrictive petals 4 and 5 are placed correspondingly at the outer and inner side of the patient's lips. The inner restrictive petal 5 is placed between the inner surface of the lips and outer surface of the front teeth of the patient. An individual match of the distances between outer and inner restrictive petals 4 and 5 depending on the patients' individual anatomical features is performed by moving of the slots 7 and 8 of the connecting element 1 and fixing it in the chosen position on the ribbed surface.

When the connecting element 1 is gripped between the front upper and lower teeth because of the step bend 2 the tongue goes down and releases the space between the palate and the tongue. The final individual regulation of the device is performed by moving the petal 4 along the ribbed surface of the connecting element 1 till this petal reaches the outer surface of the lips or the gap. This is the end of the regulation of the device and it is ready to work.

The performing of the through holes 9, 10 on the restrictive petals 4, 5 provides the opportunity of breathing through the mouth, that is the use of the present device is possible by breathing through the nose disturbances.

The presence of the springy step bend 2 on the connecting strip 1 allows improving adaptation of the device to the certain patient, for it allows regulating the position of the device depending on the anatomical features of the mouth cavity of the patient by turning the connecting element to 180°.

The presence of the lay in the form of the connecting element 1 between the lips of the patient prevents gnashing of teeth during the sleep. The pose of the patient during the sleep does not matter.

Functionally the proposed invention may be applied for preventing and treating the snoring, the syndrome of the sleep apnea and gnashing of teeth during a sleep. The systematic use of the device works out the patient's stable conditioned reflexes, which prevent or reduce appearing of the sleep apnea, and also prevents snoring and gnashing of teeth. This is how the curative effect from applying of the proposed invention is achieved. The device may also be used for prophylaxis as the trainer for the muscles of the mouth cavity at day.

1. The device for preventing and treating the snoring and sleep apnea and/or gnashing of teeth, containing joined through the connecting element the cup-shaped fixture and the inner restrictive petal with the through holes, set up on the connecting element with the opportunity of moving along it and fixing on it, characterized by that the curves made by the two mutually perpendicular section planes of the cup-shaped fixture, paralleled to the axis of the connecting element or passing through it and presenting the planes of the symmetry of the cup-shaped fixture, have different curvature.

2. The device as claimed in claim 1, characterized by that one of the curves has the form of the parabola, and the other—the form of the arc.

3. The device as claimed in claim 2, characterized by that the longitudinal axis of the connecting element in the area of its adjoining to the fixture coincides with the axis of the parabola.

4. The device as claimed in claim 1 or 2, characterized by that the connecting element has the step bend.

5. The device as claimed in any of the claims 2-4, characterized by that the longitudinal axis of the connecting element being set off relatively to the parabola.

6. The device as claimed in any of the claims 2-5, characterized by that the angle between the tangents to the outer parabolic surfaces of the cup-shaped fixture, outgoing from the point, situated on the axis of the parabola at the distance 4-7 mm from the outer surface of the cup-shaped fixture, makes 30-90°.

7. The device as claimed in any of the claims 2-6, characterized by that the connecting element is performed in the form of the strip of the rectangular cross section.

8. The device as claimed in any of the claims 2-6, characterized by that the connecting element has an oval or circular cross section.

9. The device as claimed in any of the claims 1-8, characterized by that it is provided with the outer restrictive petal, set up on the connecting element with an opportunity of moving along it and fixing on it.

10. The device as claimed in the claim 9, characterized by that the outer restrictive petal is performed with the through holes.

11. The device as claimed in any of the claims 1-9, characterized by that the dimensions and quantity of the holes in the identified petals are chosen depending on providing for the laminarity of the airflow, breathed in or out by the statistically average patient.

12. The device as claimed in any of the claims 9-11, characterized by that the square of the surface of the outer restrictive petal exceeds the square of the surface of the inner one.

13. The device as claimed in any of the claims 10-12, characterized by that the square of each of the holes in the outer restrictive petal exceeds the square of each of the holes in the inner restrictive petal.

14. The device as claimed in any of the claims 1-13, characterized by that the cup-shaped fixture and/or the inner restrictive petal, and/or the outer restrictive petal, and/or the connective element are made of elastic material.
